(19) United States
(12) Patent Application Publication
(10) Pub. No.: US 2022/0175457 A1
(43) Pub. Date: Jun. 9, 2022

(54) Endoscopic image registration system for robotic surgery

(71) Applicant: Xiaoning Huai, Sunnyvale, CA (US)

(72) Inventor: Xiaoning Huai, Sunnyvale, CA (US)

(73) Assignee: Real Image Technology Co., Ltd, Shenzhen (CN)

(21) Appl. No.: 17/676,220

(22) Filed: Feb. 20, 2022

(30) Foreign Application Priority Data
Feb. 6, 2022 (CN) 202220249273X

(51) Int. Cl. A61B 34/10; A61B 34/30; G06T 7/30

(52) U.S. Cl. CPC A61B 34/10; A61B 34/30; G06T 7/30; A61B 2034/105; G06T 2200/08; G06T 2200/24; G06T 2207/30024; G06T 2207/10068

(57) ABSTRACT

A system for endoscopic image registration, comprising a processing module, a camera module and a display module. The processing module builds a 3D spectral data module of a human body part based on preoperative 3D imageries, spectral characteristics of tissues of anatomy of the body part and of the light source; registers the module with image data captured by the camera module; generates masks on the model referencing a point cloud coupled with the image data, and displays one or more of the image data, the model before and after the registration in comparisons to assist endoscopy or guide robotic surgery.

### TECHNICAL FIELD

The invention is applied to the fields of biology and medicine, and especially to medical image processing, endoscopic image registration and automatic surgical robots.

### BACKGROUND

Endoscopy used for minimally invasive or natural orifice inspection or surgery counts on spectral image data captured by its cameras. CT, MRI, and ultrasound and other 3D imaging modalities are often used for preoperative planning or as intraoperative auxiliary data. Positions of a human body part at surgical site need to be tracked during a surgery to assist operation by a surgeon or to guide a surgical robot, wherein tracking is accomplished through fusion or registration of data from various imaging modalities. To date, prior art image registration is primarily biased on registering endoscopic images with 3D imageries obtained preoperatively or intraoperatively as reference target.

### SUMMARY OF THE INVENTION

The invention discloses an image processing system, comprising a camera module having one or more cameras or an endoscope with one or more cameras coupled by communication links, a display module having one or more display devices coupled by communication links internally and externally, a processing module with one or more processors connected with or integrated in the camera module, including instructions, program parameters and a 3D spectral data model of a body part stored in a non-volatile storage medium to be accessed by the one or more processors through communication links. The camera module is configured to capture image data of anatomy of a body part; the processing module is configured to build, retrieve or receive a 3D spectral data module of the body part; register the model and other imageries than the image data obtained preoperatively or intraoperatively, with the image data as a reference target; The processing module is further configured to, referencing the registered model, display through the display module, one or more of the image data, the imageries other than the image data, the model before and after the registration to facilitate an endoscopy or a robotic surgery. The processing module is further configured to, referencing the registered model, perform one or more of: operating a surgical robot and registering the model, along with other imageries than the image data, with a new sequence of image data captured by the camera module. The registration of the model is performed through transforming voxels of the model distinctively with respect to the positions of individual voxels or through a coordinate transform of the model based on a set of parameters. The 3D spectral model of a body part may be built through the processing module performing the following steps: obtaining data of 3D imageries of the body part; extracting, from the data, morphological features and structures of anatomy of tissue of the body part; determining or modifying a luminance value of a voxel referencing a spatial distribution of an illumination of a light source used by the camera module, spectral characteristics of the light source and spectral characteristics of the tissue, and a relative position between the light source and the body part, wherein a first luminance value of the voxel is correlated to a second luminance value of a pixel of the image data representative of a brightness of a corresponding spot of the tissue of the body part; determining or modifying the hue value of the voxel referencing the spectral characteristics of the light source and the spectral characteristics of the tissue, wherein a difference between H value in HSV color space of a first hue of the voxel and the H value of a second hue of the pixel of the image data representative of a color of a corresponding spot of the tissue of the body part is less than a threshold, the threshold depends on the spectral characteristics of the tissue. The processing module may acquire a module built elsewhere and modify the data of the module according to a system set up.

The invention discloses an image processing method relating the system above, comprising the steps of: obtaining a 3D spectral model of a body part; capturing image data of anatomy of the body part; registering the model with the image data as reference target; displaying one or more of: the image data, imageries other than the image data, the model before and after the registration; further performing endoscopy or robotic surgery referencing the registered model; capturing new image data of anatomy of the body part; registering the model with the new image data referencing the registered model.

### BRIEF DESCRIPTION OF THE DRAWINGS

FIG. 1 is an illustration of the system architecture.

FIG. 2 is an illustration of modular construction of the camera module.

FIG. 3 is an illustration of modular construction of the display module.

FIG. 4 is an illustration of modular construction of the processing module.

FIG. 5 is an illustration of system outline.

FIG. 6 is the schematics for system operation.

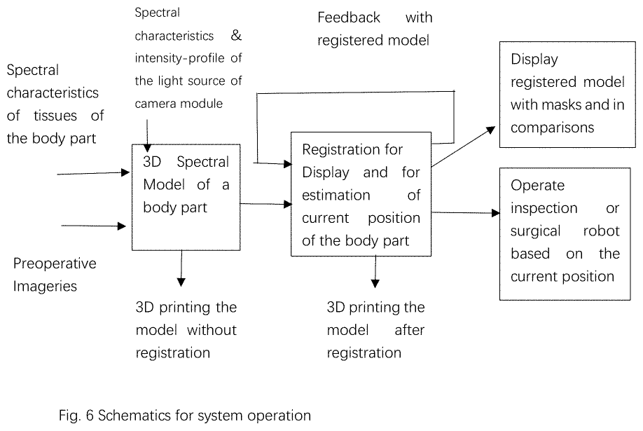

### DETAILED DESCRIPTION

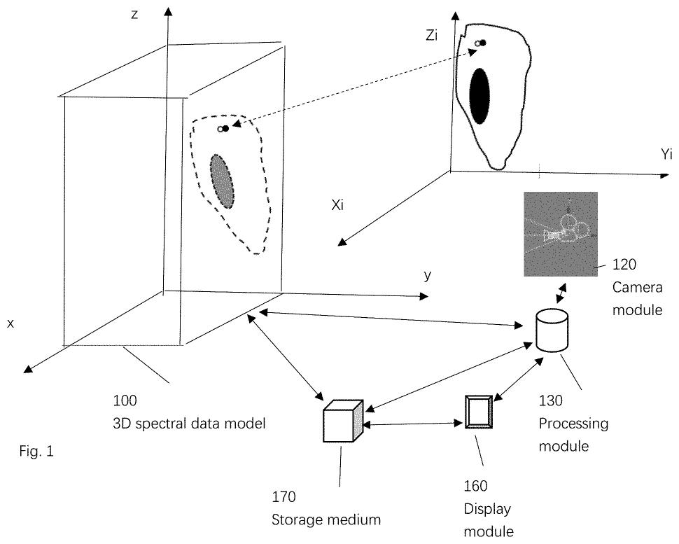

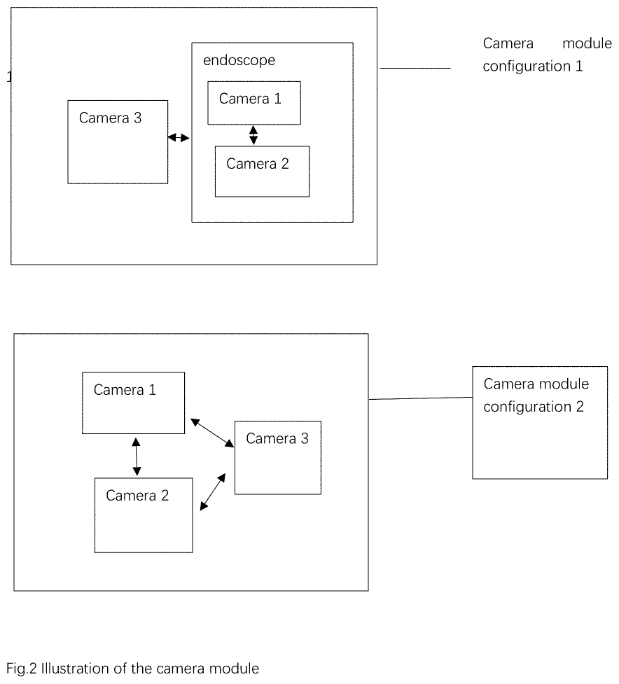

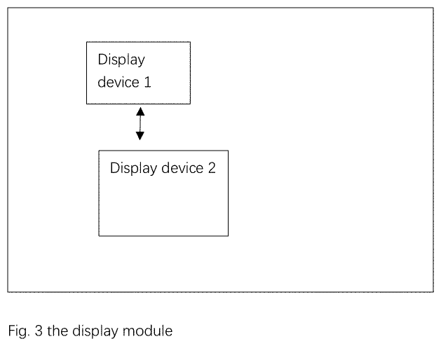

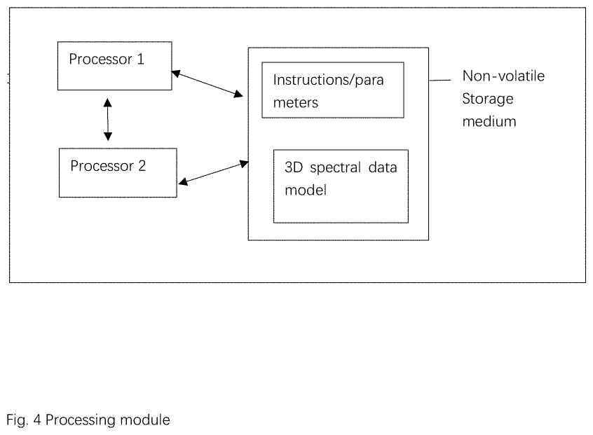

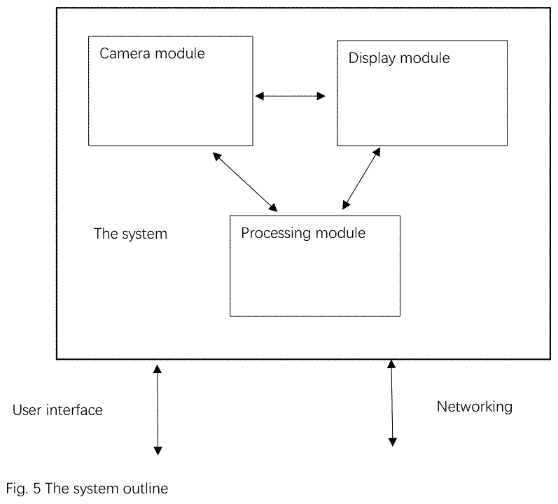

The following example embodiments are provided to illustrate the present invention without limiting the scope of the invention. The system and method disclosed in the present invention relate to the following observation and rational: The 3D imageries obtained preoperatively may not reflect the actual position of the body part in an endoscopy or a surgery. Although data of intraoperative radioactive imageries may help with dynamic positioning, their acquisition may invoke risks to the safety of patient and medical staffs as well and demand additional complexities to the operation room set up. Alternatively, or complementarily, the image data captured by cameras of an endoscope may contain pertinent real-time information of anatomy of the body part at the surgical site that may be used as an expedite reference for registering other imageries than the image data for positioning the body part. The image data comprise pixels of luminance and color values coupled with depth values, in a structure of a point cloud, of spots of a surface of anatomy of a body part. As shown in FIG. 1-FIG. 5, the system comprises a camera module having one or more cameras or an endoscope with one or more cameras, a display module having one or more display devices, a processing module with one or more processors connected with or integrated in the camera module, including instructions, program parameters and a 3D spectral data model of a body part stored in a non-volatile storage medium to be accessed by the one or more processors. The modules are connected by communication link, and the system may include user interfaces and be networked externally for control and data. The camera module is configured to capture image data of anatomy of the body part; the processing module is configured to build, receive, retrieve and refine a 3D spectral data model of a body part with respect to the light source of the camera module, receive or retrieve the image data captured by the camera module, run one or more computer programs for endoscopy or robotic surgery, receive or retrieve imageries other than the image data, register the model and other imageries than the image data, obtained preoperatively or intraoperatively, with the image data as a reference target; and is further configured to, referencing the registered model, display through the display module, one or more of the image data, the imageries other than the image data, the model before and after the registration, thereby facilitating the endoscopy or the robotic surgery. The XᵢYᵢZᵢ coordinate system represents a coordinate system of the model during preoperative planning, while the xyz coordinate system represents the intraoperative coordinate system. The smallest dots in FIG. 1 are representative of voxels of the model, the ellipses representative of features of the body part and the largest contour representative of the boundary of the body part. A human body part may be simulated as a model of three-dimensional data structure set in a coordinate system, wherein differences between individuals may manifest as anisotropic smooth expansion or compression, displacement or rotation in each of the dimensions. A general model of a body part may be applicable to a group of individuals with the same gender, race and age. A first step in building a model may comprise extracting, from preoperative 3D imageries of CT, MRI and ultrasonics, morphological structures of anatomy of tissues of a body part; wherein the tissues may include for example skin, mucous membranes, fat, nerves, fascia, muscles, blood vessels, internal organs, bones and etc., which may constitute the objects to be modeled in a data set of voxels. The next step may comprise assigning or modifying luminance and color values of each voxel, representative of a spot of a tissue, referencing the spectral characteristics of the tissue and of the light source by which the tissue is to be illuminated. Since the intensity of light wave attenuates as it propagates in space, the profile of the attenuation of a light source with respect to a view point of a camera module in a restricted space such as an operation room may be calibrated into a look up table, by which to correlate a first luminance of a voxel of the model with a second luminance of a pixel of the image data. The color of the voxel may be determined by conducting a spectral response analysis of the tissue under illumination of the light source, or simply by taking a shot by the camera module of the tissue under the illumination of the light source. The sampling rate of voxels and the spatial resolution of the model conform to the Nyquist sampling theorem.

The 3D spectral data model of a body part may preferably be represented by P(x, y, z, λᵢ, n), wherein P represents a voxel of the model; x, y, z are the coordinates of the voxel in a coordinate system; 0; 0; 0, X₀, Y₀, Z₀ are the boundary values of the model; λᵢ is a parameter structure, wherein for example if the first element represents the light point of the voxel, λ₁=(R, G, B); And so on, λ₂=(r, g, b) representing fluorescence image value; λ₃=(ρ_{c}) representing CT image value; λ₄=(ρₘ) representing MRI image value; λ₅=(ρₛ) representing ultrasound image value; λ₆ representing a mask value of a feature, such as for a point cloud of the image data and etc.; n represents a time sequence number in one instance of application. In practical application, a voxel may comprise one or more of the above λᵢ values, or other metric values not listed above.

In an endoscopic surgery, an outlier of the body part at surgical site is normally exposed in the view field first and the hierarchical structure of the anatomy is gradually developed as the operation proceeds, wherein the amount of information obtained by the endoscope is cumulatively increased. Either a surgeon or a surgical robot has to operate based on the limited information. A current position of the body part may be estimated through a fusion or registration of the model with the image data, and other imageries than the image data, and used to guide the surgery. An example for a registration may comprise the steps of: First, the boundary of the body part in the image data may be extracted automatically by the processing module running an algorithm, or by a manual marking, or by both means above. Secondly, the processing module may optionally perform, between the model, other imageries than the image data and the detected body part in the image data, a classical image matching algorithm such as the minimum mean square error in the steps of:

Step 1: acquiring a point cloud coupled with the image data, the point cloud is representative of coordinates of a surface of the anatomy of the body part captured in the image data; Step 2, obtaining light points of voxels at positions of the coordinates of the point cloud; Step 3, calculating a mean square error between pixels of the image data mapped to the point cloud and respective light points of voxels of the model, the error including based on luminance, or color, or R, G, B components; Step 4, obtaining new coordinates after a transformation of the coordinates comprising one or more of translation, rotation, and scaling; Step 5, calculating the mean square error between the pixels of the image data mapped to the point cloud and respective new light points at positions of the new coordinates of the model, and obtaining a minimum mean square error; step 6, repeating steps 4-6 by traversing parameters of the coordinate transformation, and obtaining a set of parameters comprising data of displacement, rotation, and scaling. The registration of the model may be completed by a coordinate transform based on the set of above parameters, or by position variable transforms with respect to individual voxels or pixels. The registered model or imageries other than the image data, the image data, imageries other than the image data and the model before the registration may be displayed on the one or more display devices separately or comparatively to be inspected by the surgeon. The registered model may be used as a reference to control the surgical robot. One useful presentation of the model may be to generate a first mask on a first set of voxels of the model at coordinates of a point cloud coupled with the image data before the registration and a second mask on a second set of voxels of the model at the coordinates of the point cloud after the registration, display the models separately or comparatively, and update the display for each iteration of the registration. An optional recursive Kalman filtering with the registered model may be used as auxiliary data assisting registering the model with new image data.

Estimation of the current position of the body part and mapping of the model with the image data and other imageries than the image data may additionally or alternatively be based on correlating features derived from the image data, the model and intraoperative imagery other than the image data, wherein the features may comprise lateral relationships between organs and longitudinal relationships between layers of tissues of an organ.

Alternatively, a machine learning based registration may be developed as an emulation of a surgeon exercising his or her surgical experiences. A surgeon perceives the 3D structure of a body part based on intuitive surface data of a target. The surgical robot however, may learn not only from its own practices, but also directly or indirectly from practices of its companion robots as well, and therefore excel the surgeon in speed and accuracy of the learning. An external 3D printing apparatus may be configured to be connected to the processing module and perform 3D printing of the model.

1. A system for endoscopic image processing, comprising a camera module, a display module and processing module, wherein the processing module is configured to obtain a 3D spectral data model of a body part; obtain image data of anatomy of the body part captured by the camera module; register the model with the image data as reference target; display one or more of the image data, the model before and after the registration through the display module.

2. The system of claim 1, wherein the processing module is further configured to:
extract, from 3D imageries comprising one or more of CT, MRI and ultrasonics, morphological structures of anatomy of tissues of the body part;
determine a luminance value of a voxel of the model referencing a spatial distribution of an illumination of a light source used by the camera module, spectral characteristics of the light source and spectral characteristics of the tissue, and a relative position between the light source and the body part, wherein a first luminance value of the voxel is correlated to a second luminance value of a pixel of the image data representative of a brightness of a corresponding spot of the tissue of the body part;
determine a hue value of the voxel referencing the spectral characteristics of the light source and the spectral characteristics of the tissue, wherein a difference between H value in HSV color space of a first hue of the voxel and the H value of a second hue of the pixel of the image data representative of a color of a corresponding spot of the tissue of the body part is less than a threshold, the threshold depends on the spectral characteristics of the tissue.

3. The system of claim 1, the processing module is further configured to:
retrieve or receive a 3D spectral data model of the body part;
modify a luminance value of a voxel of the model referencing a spatial distribution of an illumination of a light source used by the camera module, spectral characteristics of the light source and spectral characteristics of the tissue, and a relative position between the light source and the body part, wherein a first luminance value of the voxel is correlated to a second luminance value of a pixel of the image data representative of a brightness of a corresponding spot of the tissue of the body part;
modify a hue value of the voxel referencing the spectral characteristics of the light source and the spectral characteristics of the tissue, wherein a difference between H value in HSV color space of a first hue of the voxel and the H value of a second hue of the pixel of the image data representative of a color of a corresponding spot of the tissue of the body part is less than a threshold, the threshold depends on the spectral characteristics of the tissue.

4. The system of claim 1, the processing module is further configured to generate one or more of: a first mask on a first set of voxels of the model, before the model is registered, at coordinates of a point cloud, the point cloud is coupled with the image data, and a second mask on a second set of voxels of the model, after the model is registered, at the coordinates of the point cloud;
display one or more of: the model before and after the registration with the mask.

5. The system of claim 1, the processing module is further configured to register imageries other than the image data with the image data as reference target, and display one or more of, through the display module, the imageries before and after the registration.

6. The system of claim 1, the processing module is further configured to operate a surgical robot referencing the registered model.

7. The system of claim 1, the processing module is further configured to register the model with new image data through referencing the registered model.

8. The system of claim 1, the processing module is further configured to register the model with the image data in the steps of:
detect a boundary of the body part in the image data automatically or by a manual marking;
obtain a mapping between the model and the image data in the boundary;
transform voxels of the model distinctively with respect to the positions of the voxels based on the mapping or,
perform a coordinate transform of the model based on a set of parameters derived from the mapping, wherein the processing module is configured to derive the set of parameters through a minimum mean square error algorithm for the mapping, comprising the steps of:
Step 1: acquire a point cloud coupled with the image data, the point cloud is representative of coordinates of a surface of the anatomy of the body part captured in the image data;
Step 2: obtain light points of the voxels at positions of the coordinates of the point cloud;
Step 3, calculate a mean square error between pixels of the image data mapped to the point cloud, and respective light points of voxels of the model;
Step 4, obtain new coordinates after a transformation of the coordinates comprising one or more of translation, rotation, and scaling;
Step 5, calculate the mean square error between the pixels of the image data mapped to the point cloud and respective new light points at positions of the new coordinates of the model, and obtain a minimum mean square error;
Step 6, repeat steps 4-6 by traversing parameters for the transformation of the coordinates, and obtain the set of parameters comprising data of displacement, rotation, and scaling.

9. The system of claim 1, the processing module is further configured to estimate a current position of the body part through correlating features derived from the image data, the model and intraoperative imagery other than the image data, the features comprising lateral relationships between organs and longitudinal relationships between layers of tissues of an organ.

10. The system of claim 3, comprising a 3D printing apparatus connected to the system, the 3D printing apparatus is configured to 3D print the model.

11. An image processing method, comprising the steps of: obtaining a 3D spectral model of a body part; capturing image data of anatomy of the body part; registering the model with the image data as reference target; displaying one or more of the image data, the model before and after the registration.

12. The method of claim 11, wherein the obtaining the 3D spectral model of the body part comprising the steps of:
extracting from 3D imageries comprising one or more of CT, MRI and ultrasonics, morphological structures of anatomy of tissues of the body part;
determining or modifying a luminance value of a voxel of the model referencing a spatial distribution of an illumination of a light source used by the camera module, spectral characteristics of the light source and spectral characteristics of the tissue, and a relative position between the light source and the body part, wherein a first luminance value of the voxel is correlated to a second luminance value of a pixel of the image data representative of a brightness of a corresponding spot of the tissue of the body part;
determining a hue value of the voxel referencing the spectral characteristics of the light source and the spectral characteristics of the tissue, wherein a difference between H value in HSV color space of a first hue of the voxel and the H value of a second hue of the pixel of the image data representative of a color of a corresponding spot of the tissue of the body part is less than a threshold, the threshold depends on the spectral characteristics of the tissue.

13. The method of claim 11, wherein the obtaining the 3D spectral model of the body part further comprising the steps of:
retrieving or receiving a 3D spectral model of the body part;
modifying a luminance value of a voxel of the model referencing a spatial distribution of an illumination of a light source used by the camera module, spectral characteristics of the light source and spectral characteristics of the tissue, and a relative position between the light source and the body part, wherein a first luminance value of the voxel is correlated to a second luminance value of a pixel of the image data representative of a brightness of a corresponding spot of the tissue of the body part;
modifying a hue value of the voxel referencing the spectral characteristics of the light source and the spectral characteristics of the tissue, wherein a difference between H value in HSV color space of a first hue of the voxel and the H value of a second hue of the pixel of the image data representative of a color of a corresponding spot of the tissue of the body part is less than a threshold, the threshold depends on the spectral characteristics of the tissue.

14. The method of claim 11, wherein the registering comprises the steps of:
detecting a boundary of the body part in the image data automatically or by a manual marking;
obtaining a mapping between the model and the image data in the boundary;
transforming voxels of the model distinctively with respect to the positions of the individual voxels based on the mapping or,
performing a coordinate transform of the model based on a set of parameters derived from the mapping, wherein the mapping comprising implementing a minimum mean square error algorithm in the steps of:
Step 1: acquiring a point cloud coupled with the image data, the point cloud is representative of coordinates of a surface of the anatomy of the body part captured in the image data;
Step 2: obtaining light points of the voxels at positions of the coordinates of the point cloud;
Step 3: calculating a mean square error between pixels of the image data mapped to the point cloud, and respective light points of voxels of the model;
Step 4: obtaining new coordinates after a transformation of the coordinates comprising one or more of translation, rotation, and scaling;
Step 5: calculating the mean square error between the pixels of the image data mapped to the point cloud and respective new light points at positions of the new coordinates of the model, and obtain a minimum mean square error;
Step 6, repeating steps 4-6 by traversing parameters for the transformation of the coordinates, and obtain the set of parameters comprising data of displacement, rotation, and scaling.

15. The method of claim 11, further comprising the steps of:
estimating a current position of the body part through correlating features derived from the image data, the model and intraoperative imagery other than the image data, the features comprising lateral relationships between organs and longitudinal relationships between layers of tissues of an organ.

16. The method of claim 11, further comprising the steps of:
generating one or more of:
a first mask on a first set of voxels of the model, before the model is registered, at coordinates of a point cloud, the point cloud is coupled with the image data, and
a second mask on a second set of voxels of the model, after the model is registered, at the coordinates of the point cloud;
displaying one or more of: the model before or after the registration with the mask.

17. The method of claim 11, further comprising the steps of:
registering imageries other than the image data with the image data as reference target;
displaying one or more of: the imageries before and after the registration.

18. The method of claim 11, further comprising the step of operating a surgical robot referencing the registered model.

19. The method of claim 11, further comprising the step of: registering the model with new image data through referencing the registered model.

20. The method of claim 12, further comprising the steps of: 3D printing the model.
